(19) United States
(12) Patent Application Publication
(10) Pub. No.: US 2022/0054668 A1
(43) Pub. Date: Feb. 24, 2022

(54) LAVAGE FLUID DELIVERY DEVICE AND SYSTEMS

(71) Applicant: Carefusion 2200, Inc., San Diego, CA (US)

(72) Inventors: John Krueger, Muskego, WI (US); Michael Plishka, Lake Villa, IL (US); Christopher McGinley, Highland Park, IL (US); Daniel Zimbler, Chicago, IL (US)

(21) Appl. No.: 17/404,712

(22) Filed: Aug. 17, 2021

Related U.S. Application Data
(60) Provisional application No. 63/068,821, filed on Aug. 21, 2020.

(51) Int. Cl. A61L 2/00; A61M 35/00

(52) U.S. Cl. CPC A61L 2/0088; A61L 2202/15; A61M 35/00

(57) ABSTRACT

A system for applying a lavage fluid to a surface, the system having a body configured to house a lavage fluid and an application member in fluid communication with the body, wherein the application member has at least one nozzle, wherein the system is configured to dispense the lavage fluid with a first fluid flow force and a first fluid flow pattern, and wherein the nozzle is configured to selectably dispense the lavage fluid with a second fluid flow force and/or a second fluid flow pattern, wherein the second fluid flow force that is different from the first fluid flow force and the second fluid flow pattern is different from the first fluid flow pattern.

### CROSS-REFERENCE TO RELATED APPLICATION(S)

The present application claims benefit of U.S. Provisional Application Ser. No. 63/068,821 entitled “LAVAGE FLUID DELIVERY DEVICE AND SYSTEMS” filed Aug. 21, 2020, which is assigned to the assignee hereof and hereby expressly incorporated by reference herein.

### TECHNICAL FIELD

The present disclosure is directed to devices and systems for applying a lavage fluid to a surface.

### BACKGROUND

Currently, lavage (that is, the washing out of a body cavity, surgical cavity, or external wound with a medically acceptable fluid) is often employed to prevent contamination of an open surgical wound, which may occur for a variety of reasons such as accidental visceral entry or perforated viscus, operations complicated by gross spillage, departure from sterile technique, and/or existing, ongoing clinical infection. Lavage processes are thus often employed to provide intraoperative antiseptic wound irrigation.

The art of lavage currently embraces a wide variety of different approaches that vary based on the situation (for example, the size and shape of the cavity or wound) and on the medical practitioner performing the lavage process (for example, a practitioner's technique preference). Currently, no specific lavage technique is standard in the art, and as such, medical facilities often require numerous different lavage devices and systems to accommodate the variety of potential approaches. The presentation of such devices and systems is also sometimes a concern, as the inadvertently inappropriate use of such devices and systems (e.g., intravenously, if the device and/or system has a similar appearance to an intravenous device and/or system) could results in devastating effects.

Moreover, several drawbacks exist with current lavage practices, including insufficiencies in antiseptic fluid properties (e.g., the amount of time necessary for an antiseptic fluid to achieve an acceptable biological effect, which may be prohibitive), the risk of systemic absorption of the antiseptic fluid, adverse reactions such as anaphylaxis, peritoneal adhesions, neurotoxicity, and respiratory insufficiency, and improper dosage or contamination of the antiseptic fluid, which are sometimes prepared ad hoc by a medical practitioner performing the lavage.

There is thus a need in the art for versatile devices and systems for performing lavage processes, and in particular, devices and systems that enable medical practitioners to safely and effectively reduce contamination in surgical wounds that are susceptible to surgical site infections.

### SUMMARY

The present disclosure is directed to devices and systems for delivering a lavage fluid, such as an antiseptic solution, to a surface. The device comprises a body that is configured to house a lavage fluid, such as an antiseptic solution. The body is further configured to be in fluid communication with at least one application member, wherein the at least one application member is configured to apply the lavage fluid to a surface sufficient for a lavage process. The devices and systems may be adaptable such that a user may select from two or more different fluid flow rates, fluid flow patterns, and/or fluid flow forces, thus providing selectable control of lavage fluid delivery to a surface. The present disclosure is also directed to methods of using the devices and systems described herein.

### BRIEF DESCRIPTION OF THE DRAWINGS

FIG. 1A shows an example of a compressible body according to aspects of the present disclosure.

FIG. 1B shows an example of a collapsible body according to aspects of the present disclosure.

FIG. 2A shows an example of a body according to aspects of the present disclosure.

FIG. 2B shows an example of a body according to aspects of the present disclosure.

FIG. 3 shows an example of a body's connection portion according to aspects of the present disclosure.

FIG. 4 shows an example of a body with an outer casing according to aspects of the present disclosure.

FIG. 5 shows an example application member according to aspects of the present disclosure.

FIG. 6 shows an example system according to aspects of the present disclosure.

FIG. 7A shows an example dispensing aid according to aspects of the present disclosure.

FIG. 7B shows an example dispensing aid according to aspects of the present disclosure.

FIG. 8 shows an example system according to aspects of the present disclosure.

FIG. 9 shows an example system according to aspects of the present disclosure.

FIG. 10 shows an example system according to aspects of the present disclosure.

FIG. 11 shows an example system according to aspects of the present disclosure.

FIG. 12 shows an example system with more than one nozzle according to aspects of the present disclosure.

### DETAILED DESCRIPTION

The present disclosure is directed to devices and systems for delivering a lavage fluid, such as an antiseptic solution, to a surface. The device comprises a body that is configured to house a lavage fluid, such as an antiseptic solution. The body is further configured to be in fluid communication with at least one application member, wherein the at least one application member is configured to apply the lavage fluid to a surface sufficient for a lavage process. The devices and systems may be adaptable such that a user may select from two or more different fluid flow rates, fluid flow patterns, and/or fluid flow forces, thus providing selectable control of lavage fluid delivery. The present disclosure is also directed to methods of using the devices and systems described herein.

As used herein, the term “lavage fluid” refers to a fluid suitable for a lavage process as described herein. As used herein, “lavage” refers to the irrigation of a body cavity, a surgical cavity, and/or an external wound.

According to some aspects, the lavage fluid may comprise an antiseptic solution. As used herein, an “antiseptic solution” refers to a solution comprising at least a solvent and one or more antiseptic agents. According to some aspects, the antiseptic solution is an aqueous solution. As used herein, the term “aqueous solution” refers to a solution wherein the solvent comprises at least a majority of water. It should be understood that in some examples, the solvent may consist of water. According to some aspects, the antiseptic solution is an alcoholic solution. As used herein, the term “alcoholic solution” refers to a solution wherein the solvent comprises at least a majority of alcohol. It should be understood that in some examples, the solvent may consist of one or more alcohols. Non-limiting examples of alcohols include, but are not limited to, ethanol, isopropyl alcohol, n-propanol, and combinations thereof.

In one non-limiting example, the antiseptic agent may comprise a cationic molecule (i.e., a molecule having a positive charge), such as a cationic surfactant or a cationic biguanide derivative (i.e., a compound derived from biguanide). According to some aspects, the antiseptic agent may comprise a bis-(dihydropyridinyl)-decane derivative (i.e., a compound derived from bis-(dihydropyridinyl)-decane). According to some aspects, the antiseptic agent may comprise an octenidine salt and/or a chlorhexidine salt. According to some aspects, the antiseptic agent may comprise alexidine, octenidine dihydrochloride, chlorhexidine gluconate, or a combination thereof.

Additionally or alternatively, the antiseptic agent may comprise iodine. According to some aspects, the iodine may be provided as an iodine complex, such as povidone-iodine (PVPI), nonylphenoxy-(ethyleneoxy)-iodine, polyethylene oxy polyprop leneoxy-iodine, undecoylinium-chloride-iodine, iodine povacrylex, and combinations thereof.

Additionally or alternatively, the antiseptic agent may comprise an oxidant (i.e., an oxidizing agent). Non-limiting examples of oxidants according to the present disclosure include, but are not limited to, sodium hypochlorite, hydrogen peroxide, and combinations thereof.

The antiseptic agent may have an antimicrobial activity sufficient to provide an acceptable log reduction of microbes in a certain time period. It should be understood that as used herein, the term “microbes” may refer to any microorganism to be killed and/or removed as a result of lavage. Example microbes include bacteria, fungi, viruses, and combinations thereof.

Example bacteria include, but are not limited to, Streptococcus mutans, S. pyogenes (group A β-hemolytic streptococci), S. salivarius, S. sanguis, Staphylococcus aureus S. epidermidis, S. haemolyticus, S. hominis, S. simulans, S. saprophyticus, methicillin/oxacillin-resistant (MRSA/ORSA) and methicillin/oxacillin-susceptible Staphylococci (MSSA/OSSA), Enterococcus (e.g., E. faecalis E. faecium, and E. hirae), vancomycin-resistant Enterococcus (VRE) and vancomycin-susceptible Enterococcus (VSE), Bacteroides fragilis, Propionibacterium acnes, Clostridium difficile (spore and vegetative cells), Selenomonas, Pseudomonas aeruginosa, Escherichia coli, Burkholderia cepacia, Proteus mirabilis, Gardnerella vaginalis, Klebsiella aerogenes, K. pneumoniae, K. pneumoniae multidrug resistant (MDR), Acinetobacter baumannii, A. baumannii MDR, Achromobacter xylosoxidans. Micrococus luteus, Ralstonia pickettii, Haemophilus influenza, and Serratia marcescens

Example fungi include, but are not limited to, Aspergillus niger, Candida albicans, C. aurus, C. dubliniensis, C. glabrata (formerly Torulopsis glabrata), C. guillermondii, C. kefyr (formerly C. pseudotropicalis), C. krusei, C. lusitaniae, C. tropicalis, Epidermophyton floccosum, Microsporum gypseum, M. canis, and Trichophyton mentagrophytes

Example viruses include, but are not limited to, those having a lipid component in their outer coat or have an outer envelope such as cytomegalovirus (CMV), human immunodeficiency virus (HIV), herpes simplex virus types 1 (HSV-1) and 2 (HSV-2), influenza virus, parainfluenza virus, variola virus (smallpox virus), vaccinia, norovirus, and coronavirus

According to some aspects, the certain time period may be a period of no more than about five minutes, optionally no more than about four minutes, optionally no more than about three minutes, optionally no more than about two minutes, and optionally no more than about one minute.

According to some aspects, the certain time period may be no more than about 120 seconds, optionally no more than about 105 seconds, optionally no more than about 90 second, optionally no more than about 75 seconds, optionally no more than about 60 seconds, optionally no more than about 45 seconds, optionally no more than about 30 seconds, and optionally no more than about 15 seconds.

It should be understood that “an acceptable log reduction” may be microbe-dependent. For example, an acceptable log reduction as described herein may refer to an acceptable log reduction of one type of microbe present on a surface (e.g., present in a body cavity or at an external wound site), a combination of two more types of microbes present on a surface, or total microbes present on a surface.

According to some aspects, an acceptable log reduction may be at least about 1.0, optionally at least about 1.1, optionally at least about 1.2, optionally at least about 1.3, optionally at least about 1.4, optionally at least about 1.5, optionally at least about 1.6, optionally at least about 1.7, optionally at least about 1.8, optionally at least about 1.9, optionally at least about 2.0, optionally at least about 2.1, optionally at least about 2.2, optionally at least about 2.3, optionally at least about 2.4, optionally at least about 2.5, optionally at least about 2.6, optionally at least about 2.7, optionally at least about 2.8, optionally at least about 2.9, optionally at least about 3.0, optionally at least about 3.1, optionally at least about 3.2, optionally at least about 3.3, optionally at least about 3.4, optionally at least about 3.5, optionally at least about 3.6, optionally at least about 3.7, optionally at least about 3.8, optionally at least about 3.9, optionally at least about 4.0, optionally at least about 4.1, optionally at least about 4.2, optionally at least about 4.3, optionally at least about 4.4, optionally at least about 4.5, optionally at least about 4.6, optionally at least about 4.7, optionally at least about 4.8, optionally at least about 4.9, and optionally at least about 5.0.

According to some aspects, the antiseptic agent may be present in the antiseptic solution in a concentration sufficient to provide an acceptable log reduction of microbes in a certain time period as described herein. According to some aspects, the antiseptic agent may be present in the antiseptic solution at a concentration of between about 0.001 and 5% w/v, optionally between about 0.001 and 2.5% w/v, optionally between about 0.001 and 1% w/v, optionally between about 0.001 and 0.1% w/v, optionally between about 0.001 and 0.01% w/v, optionally between about 0.01 and 5% w/v, optionally between about 0.01 and 2.5% w/v, optionally between about 0.01 and 2% w/v, optionally between about 0.01 and 1.5% w/v, optionally between about 0.01 and 1% w/v, and optionally about 0.5% w/v.

According to some aspects, the antiseptic agent may be present in the antiseptic solution at a concentration of between about 0.1 and 0.9% w/v, optionally between about 0.2 and 0.8% w/v, optionally between about 0.3 and 0.7% w/v, and optionally between about 0.4 and 0.6% w/v.

According to some aspects, the antiseptic agent may be present in the antiseptic solution at a concentration of between about 0.1 and 1% w/v, optionally between about 0.2 and 1% w/v, optionally between about 0.3 and 1% w/v, and optionally between about 0.4 and 1% w/v.

It should be understood that according to some aspects, the lavage fluid is not necessarily an antiseptic solution as described herein and may be any medically acceptable fluid configured to perform a lavage process as described herein. In one non-limiting example, the lavage fluid may comprise a saline solution. The saline solution may comprise water and sodium chloride in a medically acceptable concentration, such as between about 0.1 and 1%, w/v, optionally about 0.45% w/v, and optionally about 0.9% w/v.

According to some aspects, the lavage fluid, such as an antiseptic solution as described herein, may comprise a visualizing aid. As used herein, the term “visualizing aid” refers to a component in a lavage fluid configured to aid in visualizing the application of the lavage fluid. Example visualizing agents include, but are not limited to, tinting agents, staining agents, and radiopaque agents. It should be understood that the visualizing agent may be the same as or different from one of the other components of the lavage fluid. For example, the antiseptic agent may function as a visualizing agent. Additionally or alternatively, the lavage fluid may comprise a visualizing agent that is disparate from the antiseptic agent.

According to some aspects, the lavage fluid may comprise a tinting agent. As used herein, the term “tinting agent” refers to a component sufficient to provide an observable color to a fluid. The tinting agent may be sufficient to allow visualization of the lavage fluid upon application to a surface. In some non-limiting examples, the tinting agent may comprise an anionic tinting agent, such as an anionic dye. The anionic dye may be any dye suitable for medical use, such as dyes approved by the Food and Drug Administration for use in food, drugs, and/or cosmetics (i.e., “D&C” or “FD&C” dyes). Example anionic dyes include, but are not limited to, FD&C Blue No. 1 (Brilliant Blue FCF), FD&C Blue No. 2 (Indigo Carmine), FD&C Green No. 3 (Fast Green FCF), FD&C Red No. 3 (Erythrosine), FD&C Red No. 40 (Allura Red), FD&C Yellow No. 5 (Tartrazine), FD&C Yellow No. 6 (Sunset Yellow FCF), D&C Yellow No. 8 (Fluorescein), D&C Orange No. 4, and combinations thereof. Combinations may be implemented to arrive at a particular color. For example, an orange tint may comprise both FD&C Red No. 40 and D&C Yellow No. 8. Additionally or alternatively, the tinting agent may comprise a chemical compound that is observable upon exposure to visible and/or non-visible light, including, but not limited to, vitamin B-12, medical honey, fluorescent polymeric nanoparticles, water soluble luminescent carbon nanodots, quinine, and combinations thereof.

According to some aspects, the lavage fluid, such as an antiseptic solution as described herein, may comprise a staining agent. As used herein, the term “staining agent” refers to a component sufficient to temporarily or permanently color a surface with which it comes in contact.

According to some aspects, the lavage fluid, such as an antiseptic solution as described herein, may comprise a radiopaque agent. As used herein, the term “radiopaque agent” refers to a component that is opaque to the radio wave and x-ray portion of the electromagnetic spectrum sufficient for visualization. In some non-limiting examples, the radiopaque agent may comprise barium, iodine, iron oxide nanoparticles, gadolinium complex nanospheres, silica nanospheres, and combinations thereof.

According to some aspects, the lavage fluid, such as an antiseptic solution as described herein, may be basic, neutral, or acidic. According to some aspects, the lavage fluid may have a pH of between about 1 and 8, optionally between about 1 and 7, optionally between about 1 and 6, and optionally between about 2 and 5.5.

According to some aspects, the lavage fluid, such as an antiseptic solution as described herein, may comprise a buffer system. As used herein, the term “buffer system” refers to a component present in a composition or solution which may provide a resistance to significant change in pH caused by a strong acid or base. A buffer system may comprise a single agent or more than one agent, such as a weak acid and its conjugate base. A buffer system may provide a resistance to a significant pH change by interacting with a strong acid or strong base in a composition or solution, thereby at least partially preventing the pH of the composition or solution from changing significantly.

Generally, a buffer system has one or more buffer ranges wherein the buffer system has the ability to provide resistance to significant pH change. When a composition or solution comprising the buffer system has a pH inside the buffer system's buffer range, the pH of the composition or solution will not change significantly with the addition of equimolar amounts of a strong acid or strong base.

The buffer range of a buffer system is related to the acid dissociation constant (Kₐ) of one or more weak acids comprised by the buffer system. The term “acid dissociation constant” refers to the equilibrium constant of a dissociation reaction of an acid. The midpoint of a buffer range for a buffer system is generally about the logarithmic measure of the acid dissociation constant (i.e., the pKₐ, equal to −log₁₀Kₐ) of a weak acid comprised by the buffer system.

According to some aspects, the lavage fluid, such as an antiseptic solution as described herein, may comprise a stabilizing agent. As used herein, the term “stabilizing agent” refers to any component that supports the stability of a lavage fluid not otherwise explicitly described herein.

The device according to the present disclosure comprises a body configured to contain a lavage fluid as described herein. According to some aspects, the body may be compressible. As used herein, the term “compressible” refers to the ability to reversibly reduce in volume without unacceptable changes, such as an unacceptable permanent change to size, to shape, and/or to one or more of the properties as described herein. According to some aspects, the body may be configured such that upon compression, at least a portion of the antiseptic agent contained therein is dispensed. It should be understood that as used herein, “dispense” (alternatively referred to as “discharge”) may refer to transferring the lavage fluid to an application member in fluid communication with the body and/or it may refer to transferring the lavage fluid from an application member to a surface.

According to some aspects, the body may be collapsible. As used herein, the term “collapsible” refers to the ability to permanently reduce in volume. For example, a collapsible body as described herein may have a first volume when a first volume of fluid is contained therein. When at least a portion of the fluid is dispensed, the collapsible body may collapse to have a second volume, the second volume being less than the first volume. It should be understood that a collapsible body will advantageously reduce the volume of waste (e.g., the volume of the body after the fluid therein has been dispensed). A collapsible body may further provide for a more efficient fluid discharge.

According to some aspects, the body may be configured to allow at least a 10% reduction in volume when compressed and/or collapsed, optionally at least a 20% reduction in volume, optionally at least a 30% reduction in volume, optionally at least a 40% reduction in volume, optionally at least a 50% reduction in volume, optionally at least a 60% reduction in volume, optionally at least a 70% reduction in volume, optionally at least a 80% reduction in volume, optionally at least a 90% reduction in volume, and optionally at least a 99% reduction in volume.

According to some aspects, the body may comprise a body material that is compatible with the lavage fluid contained therein, that is, a material that does not chemically or physically react with the lavage fluid or otherwise render the lavage fluid unfit for medical use.

According to some aspects, the body material may be sufficient to prevent unacceptable vapor or antiseptic loss from a lavage fluid contained therein over a certain period of shelf life. It should be understood that “unacceptable vapor or antiseptic loss” may be a loss that results in the lavage fluid becoming unsuitable for its intended use. Vapor or antiseptic loss may result from, for example, adsorption or absorption of the antiseptic by a material (e.g., by the body material), evaporation of solution, evaporation of a component of a solution (e.g., an antiseptic agent of an antiseptic solution), or a combination thereof. In one non-limiting example wherein the lavage fluid comprises water and iodine as described herein, the body material may be sufficient to prevent water vapor loss and/or iodine loss over a certain period of shelf life.

As used throughout this application, the term “shelf life” refers to the length of time that a product (e.g., an antiseptic solution) may be stored while remaining within the specifications required for the form, fit, and function of the product. Shelf life may be determined by measuring certain characteristics of the product that may indicate that the product is unfit for medical use. For example, shelf life may be determined by measuring the concentration of impurities in the product, the color change of the product, the concentration of insoluble particles in the product, the potency of an active agent contained by the product (e.g., an antiseptic agent), the concentration of one or more components of the product, the pH of the product, and/or the sterility of the product after storage in long-term storage conditions. As used herein, the term “long-term storage conditions” refers to environmental conditions sufficient for a product to be acceptably stored for more than 72 hours. According to some aspects, long-term storage conditions may refer to a temperature of about 25° C. and a relative humidity of about 60%. Additionally or alternatively, shelf life may be determined by measuring the concentration of impurities in the product, the color change of the product, the concentration of insoluble particles in the product, the potency of an active agent of the product, the concentration of one or more components of the product, the pH of the product, and/or the sterility of the product after storage at 37° C. and 65% relative humidity. Additionally or alternatively, shelf life may be determined by measuring the concentration of impurities in the product, the color change of the product, the concentration of insoluble particles in the product, the potency of an active agent of the product, the concentration of one or more components of the product, the pH of the product, and/or the sterility of the product after storage at between about 15 and 30° C., with excursions at a temperature of no more than about 40° C.

According to some aspects, the period of shelf life may be at least about 20 months, optionally at least about 21 months, optionally at least about 22 months, optionally at least about 23 months, optionally at least about 24 months, optionally at least about 25 months, optionally at least about 26 months, optionally at least about 27 months, optionally at least about 28 months, optionally at least about 29 months, optionally at least about 30 months, optionally at least about 31 months, optionally at least about 32 months, optionally at least about 33 months, optionally at least about 34 months, optionally at least about 35 months, optionally at least about 36 months, optionally at least about 37 months, optionally at least about 38 months, optionally at least about 39 months, and optionally at least about 40 months.

According to some aspects, the body material may be sufficient for sterilization by any known sterilization techniques useful according to the present disclosure, including moist heat sterilization (i.e., autoclaving), gas sterilization, gamma irradiation, electron-beam (e-beam) sterilization, aseptic manufacturing processes (e.g., aseptic filtration and/or blow-fill-seal operations), and combinations thereof. According to some aspects, a body material may be determined to be sufficient for sterilization if a container comprising the body material has a Sterility Assurance Level (SAL) of at least 10⁻⁶ after sterilization and provides an acceptable result upon integrity testing for the container closure after sterilization.

According to some aspects, the body material may have a sufficient mechanical strength such that the body provides an acceptable response to impact, vibration, shaking, or a combination thereof. According to some aspects, an acceptable response refers to a response compliant with ASTM D4169-16 (Standard Practice for Performance Testing of Shipping Containers and Systems), ASTM D4728-06 (Standard Test Method for Random Vibration Testing of Shipping Containers), ASTM D642-15 (Standard Test Method for Determining Compressive Resistance of Shipping Containers, Components, and Unit Loads), or any combination thereof. According to some aspects, the body material may be safe for biomedical use. For example, the body material may comply with ISO 10993 and/or with REACH requirements. According to some aspects, the body material may be sufficient to exhibit at least a portion of the characteristics described herein over a certain period of the lavage fluid's shelf life at a temperature of between about 15 and 30° C., with excursions at a temperature of no more than about 40° C. Additionally or alternatively, the body material may be sufficient to exhibit at least a portion of the characteristics described herein over a certain period of the lavage fluid's shelf life after storage at about 25° C. and 60% relative humidity. Additionally or alternatively, the body material may be sufficient to exhibit at least a portion of the characteristics described herein over a certain period of the lavage fluid's shelf life after storage at about 37° C. and 65% relative humidity.

The body material may be rigid or flexible. As used herein, the term “rigid” refers to a stiffness sufficient to resist deformation upon normal operating forces. As used herein, the term “flexible” refers to the ability to bend or compress under normal operating forces.

Example body materials include, but are not limited to, glass, plastic, paper, foil, and any combination thereof. Example plastics useful according to the present disclosure include, but are not limited to, high-density polyethylene (HDPE), low-density polyethylene (LDPE), polypropylene, polystyrene, nylon, and any combination thereof. According to some aspects, the body material may be a lined and/or coated material, such as a lined and/or coated paper.

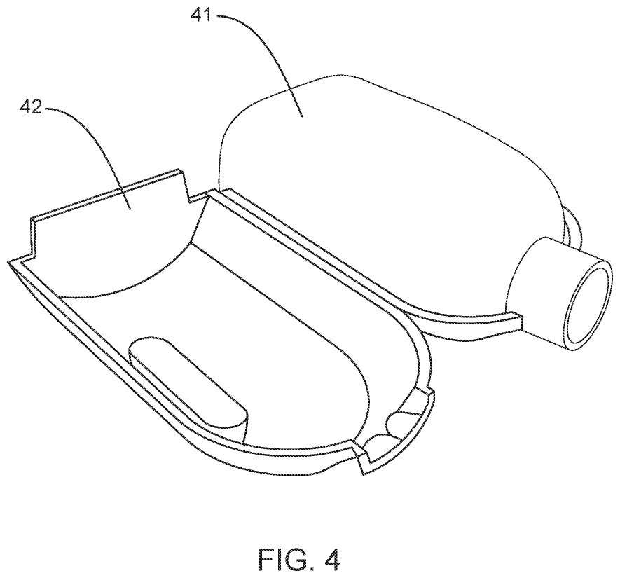

According to some aspects, the body may be provided with an outer casing. For example, FIG. 4 shows a body 41 comprising a flexible body material. Body 41 may be provided with an outer casing 42, which may be permanent or removable in relation to body 41. According to some aspects, outer casing 42 may be rigid, thus functioning to protect body 41 during storage and/or use. Outer casing 42 may additionally or alternatively function to distinguish body 41 from similar devices used in medical settings, such as intravenous (IV) fluid bags. In this way, outer casing 42 may reduce the risk of inadvertent misuse of body 41.

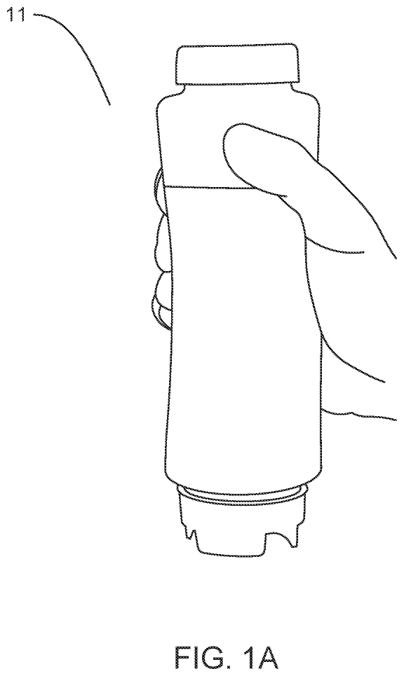

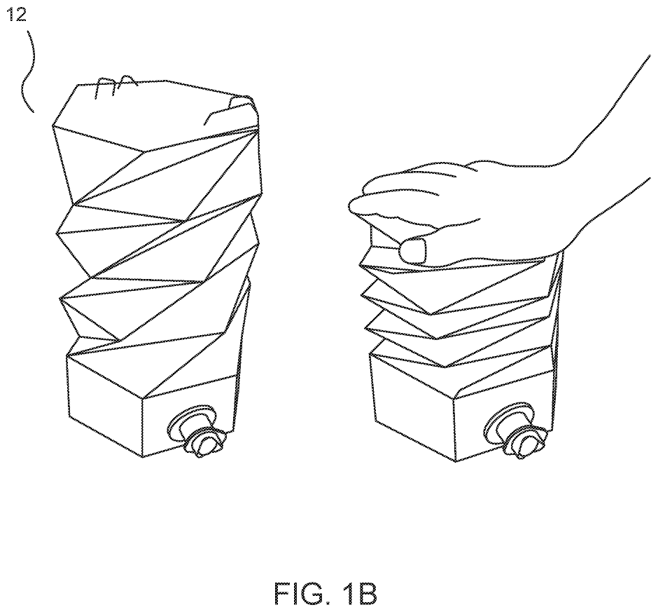

The body according to the present disclosure is configured to dispense a lavage fluid, such as an antiseptic solution, contained therein via one or more mechanisms. According to some aspects, the body may be configured to dispense the lavage fluid upon compression as described herein. For example, as shown in FIG. 1A, body 11 may be configured to dispense at least a portion of the lavage fluid contained therein in response to compression, such as squeezing. Additionally or alternatively, body 12 may be configured to dispense at least a portion of the lavage fluid contained therein in response to longitudinal compression, as shown in FIG. 1B.

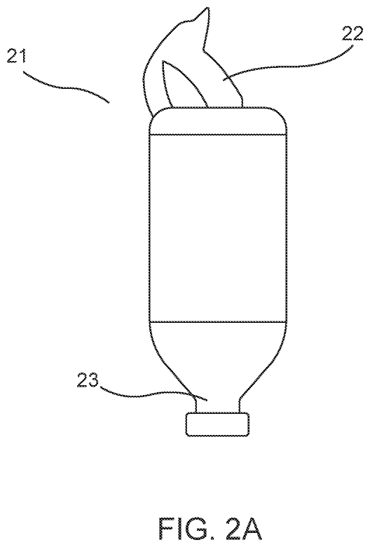

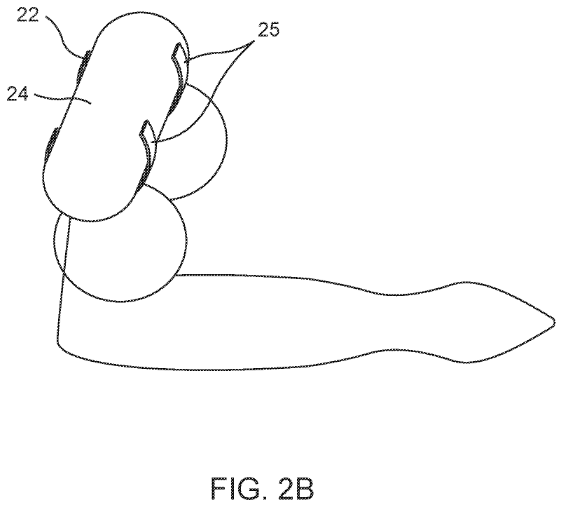

Additionally or alternatively, the body may be configured to dispense at least a portion of the lavage fluid contained therein upon orienting the body in a certain orientation. For example, as shown in FIG. 2A, body 21 may comprise an aperture 23 through which lavage fluid may be dispensed. In this example, body 21 may be configured such that when provided in a certain orientation (e.g., wherein aperture 23 is provided at or near the bottom of the body in relation to the ground), at least a portion of the lavage fluid is dispensed by the force of gravity.

In the example shown in FIG. 2A, body 21 may comprise a positioning component 22 that allows the body to be arranged in a certain orientation. The positioning component 22 may be any component configured to position and/or fix the body in a selected orientation, such as a hook, strap, snap, button, tie, or combination thereof. The positioning component 22 may be integral to the body and/or may be a separate component configured to interact with the body, such as a strap attachable to the body. The positioning component 22 may be configured to interact with a second positioning component, such as an extension arm configured to interact with a hook comprised by and/or attached to the body.

FIG. 2B shows another example of a system according to the present disclosure. In this example, body 24 is configured to interact with a separate positioning component 22, which may comprise, for example, snaps 25. In this way, body 24 may be positioned relative to and fixed to a user's arm, such as the arm of a medical practitioner performing lavage. In this example, lavage fluid may be dispensed by the force of gravity as described herein and/or by a dispensing aid as will be described herein.

According to some aspects, the body may be configured to communicate with a dispensing aid, wherein the dispensing aid is configured to provide a force sufficient to at least partially dispense the lavage fluid contained in the body. For example, the dispensing aid may comprise a pump configured to move the lavage fluid from the body. The pump may be a mechanical pump, a motorized pump, a vacuum pump, or any combination thereof.

It should be understood that the body may be configured to dispense the lavage fluid via one or a combination of the mechanisms as described herein. For example, the body may be configured to dispense the lavage fluid upon compression in conjunction with the force of gravity. Additionally or alternatively, the body may be configured to dispense the lavage fluid upon compression and/or by the force of gravity in conjunction with the force created by the pump (including, but not limited to, a vacuum force created by the pump). According to some aspects, the body may be configured to selectably dispense the lavage fluid via one or more of the mechanisms as described herein. In one non-limiting example, the body may be configured to dispense the lavage fluid upon compression both with and without the force of a pump. In this way, the user may select a desired delivery mechanism based on physical limitations (e.g., the physical capabilities of the user), a desired fluid flow force, a desired fluid flow rate, a desired fluid flow pattern (e.g., pulsed or constant), or a combination thereof.

According to some aspects, the body may be configured to dispense at least about 75% of the lavage fluid contained therein, optionally at least about 80%, optionally at least about 85%, optionally at least about 90%, optionally at least about 95%, and optionally about 100%. The body may be configured to continually dispense the lavage fluid and/or to intermittently dispense the lavage fluid. In one non-limiting example, the body may be configured to intermittently dispense the lavage fluid such that the lavage fluid is only dispensed upon compression of the body and/or upon actuation of a dispensing aid such as a pump.

The body may be configured to contain a volume of lavage fluid sufficient to perform at least a portion of a lavage process. According to some aspects, the body may be configured to contain between about 250 and 2000 mL of fluid, and optionally between about 500 and 1000 mL. According to some aspects, the body may be configured to contain about 500 mL of fluid. According to some aspects, the body may be configured to contain about 1 L of fluid.

The body according to the present disclosure may comprise a connection portion configured to selectively place the body in fluid communication with an applicator member. As used herein, the term “connection portion” refers to a portion of the body configured to provide a secure connection between the body and an application member such that fluid (e.g., an antiseptic solution) may be controllably dispensed from the body to the application member.

In one example, the connection portion is configured to fix the body and the application member such that a first aperture comprised by the body is aligned with a second aperture comprised by the application member sufficient to provide fluid communication between the body and application member. The connection portion may comprise any connection types known in the art useful according to the present disclosure.

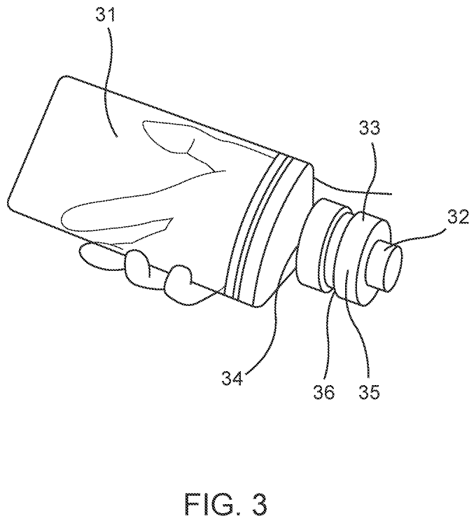

FIG. 3 shows an example of a connection portion 33 configured to connect a body 31 with an application member 32. In this example, connection portion 33 comprises protrusions configured to interact with corresponding protrusions comprised by the application member so as to form a screw connection, thereby allowing body 31 to be screwed to application member 32. It should be understood that in this example, screwing body 31 to application member 32 via connection portion 33 will align an aperture 34 of body 31 with an aperture 35 of application member 32 so as to provide fluid communication between body 31 and application member 32 when connected.

According to some aspects, the body may be provided with a removable lid, for example, a cap configured to interact with the connection portion of the body in place of the application member. It should be understood that the lid may prevent fluid discharge from the body, for example, during storage or transportation of the body.

According to some aspects, the connection portion may be provided with a fluid metering device, for example, a valve. The fluid metering device may be provided in communication with the body aperture (e.g., provided in the body aperture) sufficient to affect fluid flow from the body.

The present disclosure is also directed to a system comprising a body as described herein and one or more application members. The one or more application members may each be configured to apply a lavage fluid to a surface sufficient for a lavage process.

According to some aspects, the body may comprise a connection portion configured to interact with two or more different application members such that the system is adapted for interchanging application members. For illustrative purposes, taking the example shown in FIG. 3, the system may comprise a body 31 having a connection portion 33 as shown. The system may further comprise one or more application members each having a connection portion 36 with substantially the same size and shape such that each of the one or more application members may be interchangeably connected with body 31. In this way, a user may select from two or more application members based on lavage process preferences and requirements without requiring multiple body types. The system according to the present disclosure therefore beneficially allows a user to select from a variety of different application members, each of which may provide a unique fluid flow rate, fluid flow pattern, and/or fluid flow force, as will be described in more detail herein.

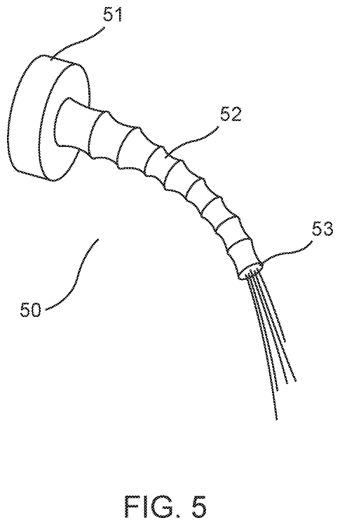

FIG. 5 shows one example application member 50 according to the present disclosure. As shown in FIG. 5, application member 50 may comprise a connection portion 51 and a discharge portion 52. Connection portion 51 may be configured to connect the application member 50 with a body as described herein. Discharge portion 52 may comprise one or more discharge apertures 53 configured to dispense a fluid (e.g., an antiseptic solution as described herein) onto a surface, such as a surgical site during a lavage process.

In the example shown in FIG. 5, discharge portion 52 may comprise a semi-flexible conduit such that the shape and/or orientation of the conduit is adjustable. In this way, the angle and/or direction of fluid discharge may be adjusted before and/or during a lavage process. As used herein, the term “semi-flexible” refers to the ability to bend or compress in addition to the ability to maintain shape when subjected to operating pressure, such as the pressure from fluid flow and/or the handling by a user. According to some aspects, the degree of flexibility of a semi-flexible component may depend at least in part on the application member material, the shape of the discharge portion, the length of the discharge portion, or a combination thereof. It should be understood that application member 50 as shown in FIG. 5 advantageously provides control of the flow path of a dispensed fluid such that a user may direct a fluid (e.g., an antiseptic solution) toward irregularly shaped and/or difficult to reach surfaces, such as irregularly shaped and/or difficult to reach surgical sites.

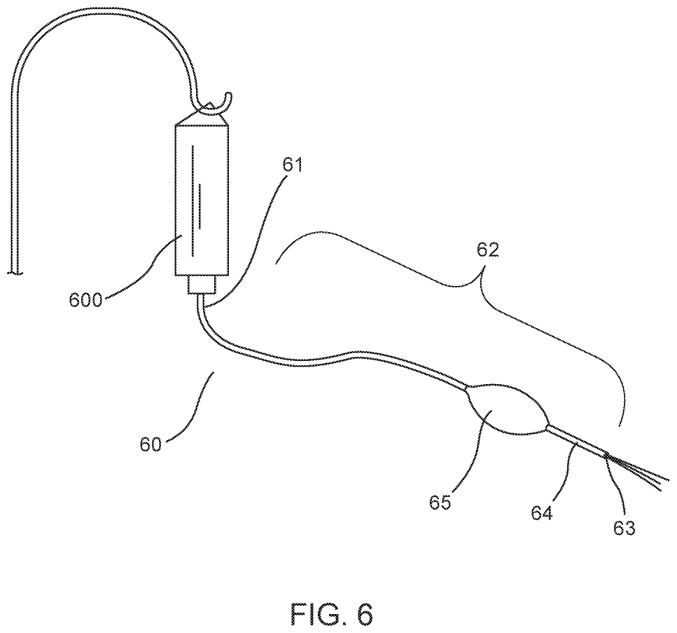

FIG. 6 shows another example application member 60 according to the present disclosure. As shown in FIG. 6, the application member 60 may comprise a connection portion 61 and a discharge portion 62. Connection portion 61 may be configured to connect application member 60 with body 600 as described herein. Discharge portion 62 may comprise one or more discharge apertures 63 configured to dispense a fluid (e.g., an antiseptic solution) onto a surface, such as a surgical site during a lavage process. It should be understood that discharge portion 62 may comprise a conduit 64 that may be a semi-flexible conduit as described in relation to FIG. 5, a flexible conduit, or a rigid conduit.

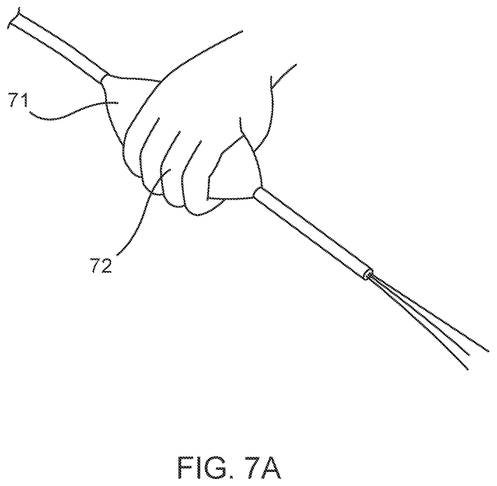

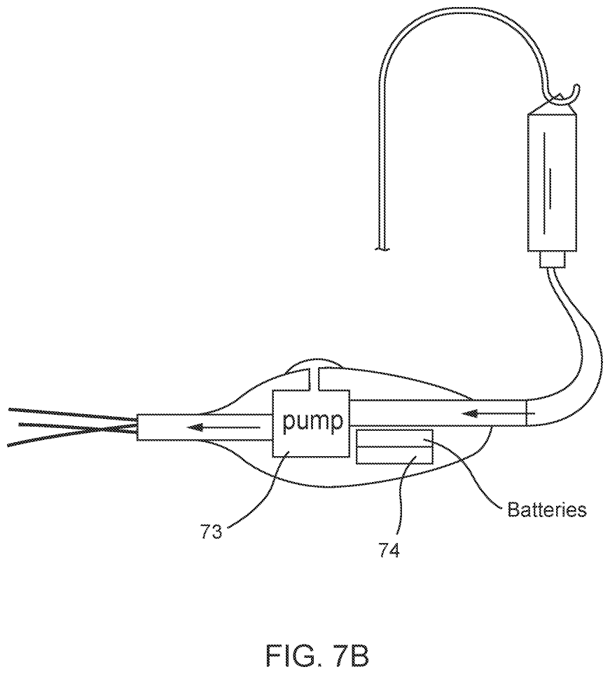

As shown in FIG. 6, application member 60 may further comprise a dispensing aid 65 as described herein, such as a pump. Dispensing aid may be a mechanical pump, for example, as shown in FIG. 7A. FIG. 7A shows a hand pump 71 that moves fluid upon compression by a user's hand 72. Additionally or alternatively, dispensing aid may be a motorized pump as shown in FIG. 7B. FIG. 7B shows a motorized pump 73 that moves fluid via electrical energy produced by, for example, batteries 74.

It should be understood that application member 60 having dispensing aid 65 as described herein may dispense a lavage fluid (e.g., an antiseptic solution) from body 600 upon actuation of dispensing aid 65 (e.g., actuation of a pump as described herein). Additionally or alternatively, dispensing aid 65 may function to dispense fluid from body 600 in conjunction with the force of gravity. For example, FIG. 6 shows an example body 600 similar to the body shown in FIG. 2A, that is, a body configured such that at least a portion of the lavage fluid contained therein is dispensed by the force of gravity when provided in a certain orientation. It should be understood that the dispensing aid will advantageously allow a user to control the fluid flow force, the fluid flow rate, and/or the fluid flow pattern (e.g., pulsed or constant) of the dispensed lavage fluid.

While the examples shown in FIGS. 5 and 6 show discharge portions having one discharge aperture, it should be understood that the discharge portion may comprise two, three, four, or more discharge apertures. Each of the discharge apertures may be the same size as or a different size from one or more of the other discharge apertures. Additionally or alternatively, each of the discharge apertures may have the same shape as or a different shape from one or more of the other discharge apertures. The shape and/or size of the one or more discharge apertures may be selected to provide a certain fluid flow force, fluid flow rate, and/or fluid flow pattern. According to some aspects, the shape and/or size of the one or more discharge apertures may be adjustable such that the fluid flow force, fluid flow rate, and/or fluid flow pattern of a dispensed fluid may be adjustable.

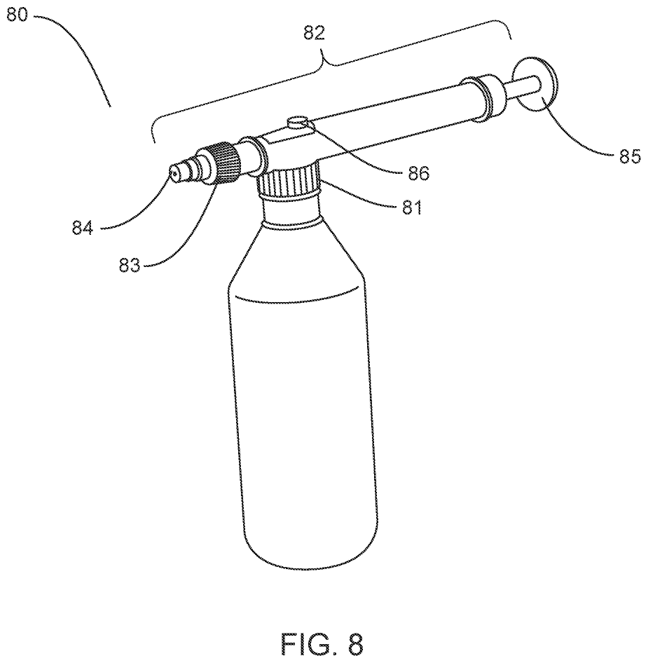

According to some aspects, the one or more discharge apertures may be provided in a nozzle portion of the discharge portion of an application member. For example, FIG. 8 shows a body 800 in fluid communication with an application member 80 having a connection portion 81 and a discharge portion 82 as described herein. As shown in FIG. 8, discharge portion 82 may comprise a nozzle 83 having one or more discharge apertures 84 as described herein. It should be understood that nozzle 83 may be removable and replaceable, thereby allowing the same application member 80 to interchangeably comprise at least two different nozzles 83. The system according to the present disclosure may thus comprise at least one application member and two or more interchangeable nozzles as described herein.

In the example shown in FIG. 8, application member 80 may further comprise a dispensing aid comprising a pump shaft 85 and an actuator 86, such as a button. In this example, nozzle 83 may be adapted to provide a mist of fluid in conjunction with pump shaft 85 upon actuation of actuator 86 by any mechanism known in the art. It should be understood that nozzle 83 may additionally or alternatively be configured to provide a stream of fluid, a spray of fluid, or a combination thereof.

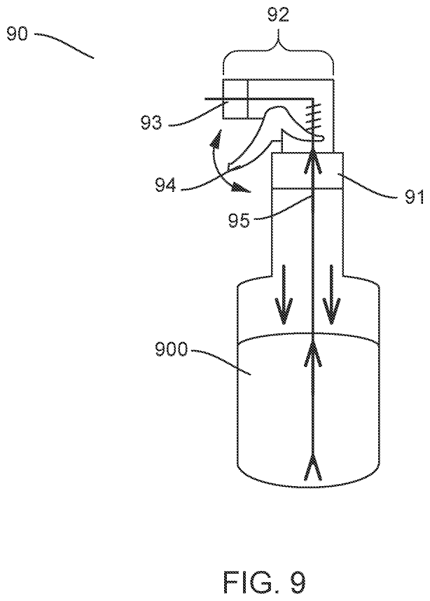

FIG. 9 shows another example of a system according to the present disclosure. As shown in FIG. 9, application member 90 may comprise a connection portion 91 and a discharge portion 92 as described herein. Discharge portion may comprise a nozzle 93 and an actuator 94, such as a trigger. In this example, application member 90 may further comprise a conduit 95 in fluid communication with a fluid contained in body 900 as described herein. In this example, body 900 may be pressurized. Upon actuation of actuator 94 (such as compressing the trigger), pressure in conduit 95 may drop below the pressure of body 900, thus forcing fluid from body 900 unto application member 90. Nozzle 93 may be configured to provide, for example, a fluid stream, a fluid mist, a fluid spray, or a combination thereof.

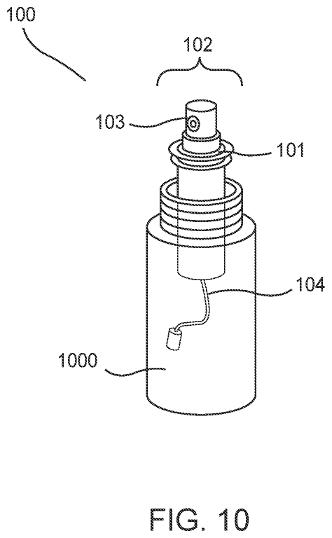

FIG. 10 shows another example of a system according to the present disclosure. As shown in FIG. 10, application member 100 may comprise a connection portion 101 and a discharge portion 102 as described herein. Discharge portion may comprise a nozzle 103. In this example, nozzle 103 may also function as an actuator, for example, by pressing nozzle 103 toward body 1000. Application member 100 may further comprise a conduit 104 in fluid communication with a fluid contained in body 1000. As described in relation to FIG. 9, body 1000 may be pressurized. Upon actuation of nozzle 103, pressure in conduit 104 may drop below the pressure of body 1000, thus forcing fluid from body 1000 unto application member 100, as described in relation to FIG. 9.

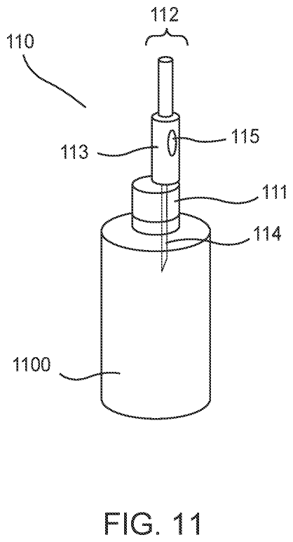

FIG. 11 shows another example system according to the present disclosure, including an application member 110, connection portion 111, discharge portion 112 including nozzle 113, and conduit 114, similar to the example shown in FIG. 10. FIG. 11 shows that nozzle 113 may further comprise an actuator 115, such as a button. As described in relation to FIGS. 9 and 10, body 1100 may be pressurized such that, upon actuation of actuator 115 (i.e., by pressing the button), pressure in conduit 114 may drop below the pressure of body 1100, thus forcing fluid from body 1100 into application member 110.

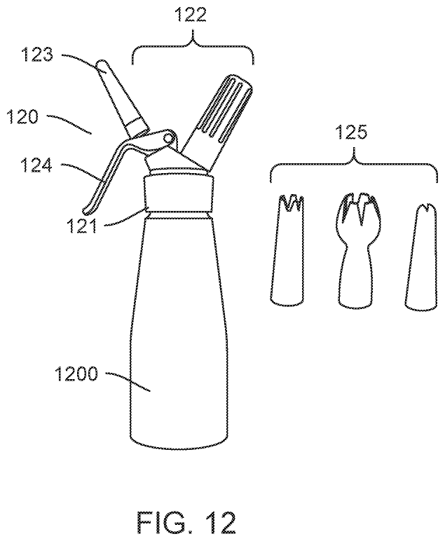

FIG. 12 shows another example of a system according to the present disclosure. As shown in FIG. 12, application member 120 may comprise a connection portion 121 and a discharge portion 122 as described herein. Discharge portion may comprise a first nozzle 123 and an actuator 124. As described in relation to FIGS. 9, 10, and 11, body 1200 may be pressurized. Additionally or alternatively, the system may comprise a cartridge containing a propellant (not shown) configured to provide an aerosol as known in the art. The propellant may be any propellant acceptable for medical use according to the present disclosure, including, but not limited to, carbon dioxide, nitrous oxide, nitrogen, helium, argon, air, and any combination thereof. As used herein, the term “air” refers to the natural atmosphere of the Earth.

The example system shown in FIG. 12 also shows a plurality of interchangeable nozzles 125 as described herein. It should be understood that each of the plurality of interchangeable nozzles 125 is configured to be interchangeable with nozzle 123, thus providing a single application member 120 and body 1200 configured to dispense a fluid to a surface via a variety of nozzles so as to provide a variety of different, selectable fluid flow rates, fluid flow patterns, and/or fluid flow forces, as described herein.

It should be understood that the systems described herein may comprise at least a body configured to be in fluid communication with one or more different application members, each of the one or more different applications members having at least one discharge aperture, wherein the at least one discharge aperture is optionally comprised by a removable and replaceable nozzle, as described herein. It should be understood that the systems as described herein may thus be configured to deliver a lavage fluid to a surface via one or more different, selectable fluid flow rates, fluid flow patterns, and/or fluid flow forces, as described herein.

For example, the system may comprise at least two different application members and/or at least two different nozzles as described herein, wherein each of the at least two different application members and/or at least two different nozzles are configured to provide a unique fluid flow rate, fluid flow pattern, and/or fluid flow force. According to some aspects, a single application member and/or a single nozzle may be configured to provide at least two unique fluid flow rates, fluid flow patterns, and/or fluid flow forces, such as by providing one or more discharge apertures with adjustable shapes and/or sizes, as described herein.

According to some aspects, the system may be configured to provide an acceptable fluid flow rate for a lavage process. As used herein, the term “fluid flow rate” refers to the rate at which a fluid is applied to a surface, such as to a human subject during a lavage process. The fluid flow rate may depend at least partially on the delivery mechanism (e.g., compressing the body, orientating the body, utilizing a dispensing aid, or a combination thereof, as described herein) and/or the properties of the application member and/or nozzle as described herein. According to some aspects, a fluid flow rate may be related to a fluid flow force. For example, an increased fluid flow rate may correspond with an increased fluid flow force, and vice versa. The system according to the present disclosure may be configured to provide at least two different, selectable fluid flow rates, optionally at least three, optionally at least four, and optionally at least five.

According to some aspects, the system may be configured to provide an acceptable fluid flow force for a lavage process. As used herein, the term “fluid flow force” refers to the force of a fluid acting on a surface, such as on a human subject during a lavage process. An acceptable fluid flow force may be determined based on the lavage process requirements. Example fluid flow forces useful according to the present disclosure include, but are not limited to, between about 10 and 50 g, and optionally between about 15 and 45 g. According to some aspects, the fluid flow force may be about 15 g. According to some aspects, the fluid flow force may be between about 30 and 45 g. Other example fluid flow forces useful according to the present disclosure include, but are not limited to, between about 1 and 15 psi (referred to herein as “low pressure”) and between about 35 and 70 psi (referred to herein as “high pressure”).

It should be understood that the fluid flow force provided by the systems as described herein may depend at least partially on the delivery mechanism and/or the properties of the application member and/or nozzle as described herein. The system according to the present disclosure may be configured to provide at least two different, selectable fluid flow forces, optionally at least three, optionally at least four, and optionally at least five. It should be understood that each of the selectable fluid flow forces may correspond with, for example, a specific delivery mechanism, a specific application member, a specific nozzle, or a combination thereof, as described herein. For example, one or more selectable flow forces may correspond with an application member having an actuator as described herein, such as a trigger, wherein each of the one or more selectable flow forces may correspond with a degree of trigger compression. In another example, one or more selectable flow forces may correspond with a nozzle having one or more discharge apertures, wherein each of the one or more selectable flow forces may correspond with the shape and/or size of the one or more discharge apertures.

According to some aspects, the system is configured to provide an acceptable fluid flow pattern for a lavage process. As used herein, the term “fluid flow pattern” refers to the pattern with which a fluid is dispensed from a device and/or applied to a surface, such as to a human subject during a lavage process. In some non-limiting examples, the fluid flow pattern may comprise a fluid mist (i.e., a suspension of finely divided fluid in a gas), a fluid stream (i.e., a steady succession of fluid), a fluid spray (i.e., finely divided fluid), or a combination thereof. The fluid flow pattern may be constant (e.g., fluid continually dispensed from a device and/or applied to a surface) or pulsed (e.g., the fluid intermittently dispensed from a device and/or applied to a surface).

A fluid flow pattern may additionally or alternatively refer to the angle at which a fluid flow path is dispensed from a device and/or applied to a surface. For example, a fluid flow path may have a fluid flow pattern that is about perpendicular to a longitudinal axis of a body as described herein.

Additionally or alternatively, a fluid flow pattern may refer to the geometric shape of a fluid path. It should be understood that the geometric shape of a fluid path refers to a shape defined by the cross-sectional view of a fluid flow path in any of the x-direction, y-direction, and z-direction.

It should be understood that the fluid flow pattern may depend at least in part on the delivery mechanism and/or the application member and/or nozzle as described herein. The system according to the present disclosure may be configured to provide at least two different, selectable fluid flow patterns, optionally at least three, optionally at least four, and optionally at least five. For example, one or more selectable flow patterns may correspond with a dispensing aid such as a pump, wherein the pump may be configured to provide a constant flow of fluid from the body and/or to provide a pulsed flow of fluid from the body. In another example, one or more selectable flow patterns may correspond with the application member's discharge portion, such as a discharge portion comprising a semi-flexible conduit as described herein. In this example, the one or more selectable flow patterns may comprise one or more fluid delivery angles corresponding with the shape and/or orientation of the semi-flexible conduit as described herein.

According to some aspects, one or more components of the system described herein may be provided in sterile packaging. As used herein, the term “sterile packaging” refers to packaging that provides a sterile environment so as to maintain sterility of a contained sterile product. Example sterile packaging includes, but is not limited to, sterile blister packaging, sterile safe-edge trays, sterile surgical trays, sterile customized thermoforms, and combinations thereof. It should be understood that one or more components of the system may be provided in the same sterile packaging and/or separate sterile packaging from at least one other component of the system. For example, a first component of the system may be contained in a first sterile packaging and a second component of the system may be contained in a second sterile packaging. In one non-limiting example, the system may comprise a body contained in a first sterile packaging and an application member contained in a second sterile packaging. It should be understood that providing one or more components of the system in different sterile packaging allows for the removal of each component of the system immediately prior to its use, thus preventing one or more components from prolonged exposure to an unsterile environment. In this way, a fully assembled sterile presentation of the system may be achieved.

The present disclosure is also directed to methods of using the devices and systems described herein. For example, the method may comprise providing a body containing a lavage fluid, wherein the body comprises a connection portion. The method may comprise placing the body in fluid communication with an application member and dispensing the lavage fluid as described herein sufficient to perform a lavage process.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

The word “about” is used herein to mean within ±5% of the stated value, optionally within ±4%, optionally within ±3%, optionally within ±2%, optionally within ±1%, optionally within ±0.5%, optionally within ±0.1%, and optionally within ±0.01%.

What is claimed is:

1. A system for applying a lavage fluid to a surface, the system comprising:
a body configured to house a lavage fluid, and
an application member in fluid communication with the body, wherein:
the application member comprises a nozzle configured to dispense the lavage fluid,
the nozzle is configured to dispense the lavage fluid with a first fluid flow force and a second fluid flow pattern, and
wherein the nozzle is configured to selectably dispense the lavage fluid with a second fluid flow force and/or a second fluid flow pattern, wherein the second fluid flow force is different from the first fluid flow force and the second fluid flow pattern is different from the first fluid flow pattern.

2. The system according to claim 1, wherein the nozzle comprises one or more discharge apertures with an adjustable size,
wherein a first size of the one or more discharge apertures corresponds with the first fluid flow force and the first fluid flow pattern, and
wherein a second size of the one or more discharge apertures corresponds with the second fluid flow force and/or the second fluid flow pattern.

3. The system according to claim 1, wherein the nozzle comprises one or more discharge apertures with an adjustable shape,
wherein a first shape of the one or more discharge apertures corresponds with the first fluid flow force and the first fluid flow pattern, and
wherein a second shape of the one or more discharge apertures corresponds with the second fluid flow force and/or the second fluid flow pattern.

4. The system according to claim 1, wherein the nozzle is configured to selectably dispense the lavage fluid as at least two of a fluid stream, a fluid mist, and a fluid spray.

5. The system according to claim 1, wherein the body is compressible.

6. The system according to claim 5,
wherein the system further comprises a pump configured to provide a force sufficient to at least partially dispense the lavage fluid, and
wherein the body is configured to dispense the lavage fluid upon compression of the body both with and without the force of the pump.

7. The system according to claim 1, wherein the body is configured to dispense at least a portion of the lavage fluid upon orienting the body in a first orientation.

8. The system according to claim 7,
wherein the system further comprises a pump configured to provide a force sufficient to at least partially dispense the lavage fluid, and
wherein the body is configured to dispense the lavage fluid upon orienting the body in the first orientation both with and without the force of the pump.

9. The system according to claim 1, wherein the body is collapsible.

10. The system according to claim 1, wherein the lavage fluid comprises an antiseptic agent and water, the antiseptic agent comprising iodine, an octenidine salt, a chlorhexidine salt, alexidine, an oxidant, or a combination thereof.

11. A system for applying a lavage fluid to a surface, the system comprising:
a body configured to house a lavage fluid; and
an application member in fluid communication with the body,
wherein:
the application member comprises at least two interchangeable nozzles configured to dispense the lavage fluid, and
wherein the at least two interchangeable nozzles comprises a first nozzle configured to dispense the lavage fluid with a first fluid flow force and a second nozzle configured to dispense the lavage fluid with a second fluid flow force that is different from the first fluid flow force.

12. The system according to claim 11, wherein the body is compressible.

13. The system according to claim 12,
wherein the system further comprises a pump configured to provide a force sufficient to at least partially dispense the lavage fluid, and
wherein the body is configured to dispense the lavage fluid upon compression of the body both with and without the force of the pump.

14. The system according to claim 11, wherein the body is collapsible.

15. The system according to claim 11, wherein the body is configured to dispense at least a portion of the lavage fluid upon orienting the body in a first orientation.

16. The system according to claim 15,
wherein the system further comprises a pump configured to provide a force sufficient to at least partially dispense the lavage fluid, and
wherein the body is configured to dispense the lavage fluid upon orienting the body in the first orientation both with and without the force of the pump.

17. The system according to claim 11, wherein the first application member comprises:
a connection portion configured to connect the first application member with the body sufficient to provide fluid communication between the first application member and the body, and
a discharge portion selectably connectable with the at least two interchangeable nozzles,
wherein the discharge portion comprises a semi-flexible conduit.

18. The system according to claim 11, wherein the second application member comprises:
a conduit,
a connection portion configured to connect the second application member with the body sufficient to provide fluid communication between the second application member and the body via the conduit,
an actuator, and
a discharge portion selectably connectable with the at least two interchangeable nozzles,
wherein the system is configured to dispense the lavage fluid upon actuation of the actuator.

19. The system according to claim 18, wherein the system comprises a cartridge containing a propellant.

20. The system according to claim 11, wherein the lavage fluid comprises an antiseptic agent and water, the antiseptic agent comprising iodine.
